(19) United States
(12) Patent Application Publication
(10) Pub. No.: US 2026/0015562 A1
(43) Pub. Date: Jan. 15, 2026

(54) MICROFLUIDIC DEVICES CONTAINING HYDROGELS, AND TECHNIQUES FOR MAKING AND USING

(71) Applicant: Xellar Ltd., Grand Cayman (KY)

(72) Inventors: Xin Xie, Lexington, MA (US); Ming Pan, Somerville, MA (US); Xiaohua Qian, Sudbury, MA (US)

(73) Assignee: Xellar Ltd., Grand Cayman (KY)

(21) Appl. No.: 19/092,065

(22) Filed: Mar. 27, 2025

Related U.S. Application Data
(63) Continuation of application No. PCT/US2023/025749, filed on Jun. 20, 2023.
(60) Provisional application No. 63/412,174, filed on Sep. 30, 2022.
(60) Provisional application No. 63/412,273, filed on Sep. 30, 2022.
(60) Provisional application No. 63/412,279, filed on Sep. 30, 2022.
(60) Provisional application No. 63/437,954, filed on Jan. 9, 2023.
(60) Provisional application No. 63/437,955, filed on Jan. 9, 2023.

(51) Int. Cl. C12M 3/06; C12M 1/00; C12M 1/12; C12M 1/34

(52) U.S. Cl. CPC C12M 23/16; C12M 23/58; C12M 25/14; C12M 29/10; C12M 41/36

(57) ABSTRACT

The present disclosure generally relates to microfluidic devices, which may contain hydrogels in certain embodiments. In some aspects, a hydrogel or other scaffold medium may be present within a first microfluidic channel, and cells that are present may be imaged. The cells may be kept alive by exposure to cell media, which may be supplied via a second microfluidic channel. The first and second microfluidic channels may meet at a common interconnect region, in which the hydrogel can be directly exposed to the cell media, and nutrients, dissolved gases, waste, etc., can pass from the media to the cells or vice versa, e.g., through the hydrogel. In addition, in some cases, a polymer may be present between the hydrogel and the microfluidic channel, e.g., to position the hydrogel relative to one or more walls of the microfluidic channel. Other embodiments are generally directed to methods of making or using such devices, kits using such devices, or the like.

### RELATED APPLICATIONS

This application claims the benefit of U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.” Each of these is incorporated herein by reference in its entirety.

### FIELD

The present disclosure generally relates to microfluidic devices, which may contain hydrogels in certain embodiments.

### BACKGROUND

The human body is a holistic system, its biology multi-faceted and intricately interconnected. Traditional drug discovery and development efforts have relied on simplified models and reductionistic tools to discover and test new drugs.

For example, in vitro cell culture has been an integral part of the drug discovery and development process. Over the years, studies with 2D cell cultures have provided valuable information on drug candidates through preclinical phases of the development process, from target ID and efficacy, to lead optimization, candidate selection, and the like. Regulatory bodies such as the FDA rely on cell culture data to gain insight into the efficacy, safety, and mechanisms of action of drug candidates.

However, despite wide-ranging applications, conventional 2D cell cultures are known to have various limitations, because cells grown on flat surfaces or petri dishes lack the relevant cellular microenvironment therefore cannot fully emulate physiological cell functions in human body. There is thus a great need for in vitro cell models that closely mimic cell functions in vivo.

### SUMMARY

The present disclosure generally relates to microfluidic devices, which may contain hydrogels in certain embodiments. The subject matter of the present disclosure involves, in some cases, interrelated products, alternative solutions to a particular problem, and/or a plurality of different uses of one or more systems and/or articles.

In one aspect, the present disclosure is generally directed to an article. In one set of embodiments, the article comprises a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets; a hydrogel filling the first microfluidic channel but not the second microfluidic channel; and a polymer positioned between a wall of the first microfluidic channel and the hydrogel.

In another set of embodiments, the article comprises a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets; a hydrogel partially filing the common interconnect region such that the hydrogel does not prevent bulk fluid flow from the first inlet to the first outlet; and a polymer positioned between the hydrogel and a wall of the common interconnect region.

According to yet another set of embodiments, the article comprises a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets and defining a channel axis; a hydrogel partially filing the common interconnect region such that at least 20% of any cross-section of the common interconnect region relative to the channel axis is not filled with the hydrogel; and a polymer positioned between the hydrogel and a wall of the common interconnect region.

In still another set of embodiments, the article comprises a substrate defining a first microfluidic channel having an inlet and an outlet, and a second microfluidic channel having an inlet and an outlet, the first microfluidic channel and the second microfluidic channel positioned parallel within a common interconnect region positioned between their respective inlets and outlets; a scaffold medium filling the first microfluidic channel but not the second microfluidic channel; and a polymer positioned between the scaffold medium and the first microfluidic channel.

In still another set of embodiments, the article comprises a substrate defining a microfluidic channel defining a channel axis from an inlet to an outlet of the microfluidic channel therein, a hydrogel partially filing the microfluidic channel such that the hydrogel does not prevent bulk fluid flow from the inlet to the outlet, and a polymer positioned between the hydrogel and a microfluidic channel wall.

The article, in yet another set of embodiments, comprises a substrate defining a microfluidic channel defining a channel axis, a hydrogel partially filing the microfluidic channel such that at least 20% of any cross-section of the microfluidic channel relative to the channel axis is not filled with the hydrogel, and a polymer positioned between the hydrogel and a microfluidic channel wall.

Another aspect is generally drawn to a method. According to one set of embodiments, the method comprises imaging living cells in contact with a hydrogel contained within a microfluidic channel defined by a substrate, and flowing cell media through the microfluidic channel containing the hydrogel. In some embodiments, the hydrogel partially fills the microfluidic channel and is positioned relative to the microfluidic channel by a polymer.

In another set of embodiments, the method comprises imaging living cells contained within a plurality of regularly arranged repeat units defined by a substrate. In some cases, at least one repeat unit containing a first microfluidic channel having an inlet and an outlet, and a second microfluidic channel having an inlet and an outlet. In certain embodiments, the first microfluidic channel and the second microfluidic channel positioned parallel within a common interconnect region positioned between their respective inlets and outlets. In some instances, the imaged living cells are contained within the common interconnect region.

In yet another set of embodiments, the method comprises imaging living cells contained within a plurality of regularly arranged repeat units defined by a substrate. In some cases, at least one repeat unit contains a microfluidic channel partially filled with a hydrogel. The hydrogel may be in contact with the imaged living cells. In some cases, a polymer may be positioned between the first microfluidic channel and the hydrogel.

The method, in still another set of embodiments, comprises imaging living cells in contact with a hydrogel contained within a microfluidic channel defined by a substrate. In some cases, the microfluidic channel may define a channel axis. In certain embodiments, the microfluidic channel is partially filled with the hydrogel such that the hydrogel does not prevent bulk fluid flow from the inlet to the outlet. In some cases, a polymer may be positioned between the first microfluidic channel and the hydrogel.

In yet another set of embodiments, the method comprises imaging living cells in contact with a hydrogel contained within a microfluidic channel defined by a substrate. In some cases, the microfluidic channel may define a channel axis. In some embodiments, the microfluidic channel is partially filled with the hydrogel such that at least 20% of any cross-section of the microfluidic channel relative to the channel axis is not filled with the hydrogel. In some cases, a polymer may be positioned between the first microfluidic channel and the hydrogel. In addition, in some cases, less than 80% of any cross-section of the microfluidic channel relative to the channel axis is not filled with the hydrogel.

The method, in still another set of embodiments, comprises imaging living cells in contact with a scaffold medium contained within a microfluidic channel defined by a substrate, and flowing cell media through the microfluidic channel containing the scaffold medium. In some embodiments, the scaffold medium partially fills the microfluidic channel. In some cases, a polymer may be positioned between the first microfluidic channel and the scaffold medium.

In another aspect, the present disclosure encompasses methods of making one or more of the embodiments described herein, for example, microfluidic devices containing hydrogels. In still another aspect, the present disclosure encompasses methods of using one or more of the embodiments described herein, for example, microfluidic devices containing hydrogels.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates microfluidic channels meeting at a common interconnect region, in accordance with certain embodiments;

FIG. 2 illustrates a microfluidic chip having four repeat units, in another embodiment;

FIGS. 3A-3B illustrate microfluidic chips having the physical form of a microscope slide, in yet other embodiments;

FIG. 4 illustrates a layout of repeat units able to match the spacing between wells in a standard 384 microwell plate, according to still another embodiment;

FIGS. 5-6 illustrate barrierless separation between a hydrogel containing cells (bottom) and cell media (top), in yet other embodiments;

FIG. 7 illustrates a common interconnect region having three microfluidic channels, in yet another embodiment; and

FIG. 8 illustrates microfluidic channels meeting at a common interconnect region, in accordance with certain embodiments.

### DETAILED DESCRIPTION

The present disclosure generally relates to microfluidic devices, which may contain hydrogels in certain embodiments. In some aspects, a hydrogel or other scaffold medium may be present within a first microfluidic channel, and cells that are present may be imaged. The cells may be kept alive by exposure to cell media, which may be supplied via a second microfluidic channel. The first and second microfluidic channels may meet at a common interconnect region, in which the hydrogel can be directly exposed to the cell media, and nutrients, dissolved gases, waste, etc., can pass from the media to the cells or vice versa, e.g., through the hydrogel. In addition, in some cases, a polymer may be present between the hydrogel and the microfluidic channel, e.g., to position the hydrogel relative to one or more walls of the microfluidic channel. Other embodiments are generally directed to methods of making or using such devices, kits using such devices, or the like.

For example, certain aspects as discussed herein are generally drawn to microfluidic devices that can contain cells, e.g., in contact with a hydrogel or another scaffold medium. For example, cells may be cultured within a microfluidic device, e.g., on or in a hydrogel. The cells may thus be cultured within such a device in an environment that is more similar to their native environment (e.g., where the hydrogel or other scaffold medium may act as an extracellular matrix). In some cases, cells cultured in such conditions may exhibit more physiologically relevant behavior, e.g., due to improved or more biologically relevant cell-to-cell or cell-to-environment interactions. In addition, in certain embodiments, the cells may be cultured in a manner as to emulate various functions of specific organs, e.g., the microfluidic device may be used as an organ-on-a-chip device.

In some embodiments, a hydrogel or another scaffold medium may be contained within a microfluidic device, e.g., within a microfluidic channel defined in a substrate forming the microfluidic device. The hydrogel (or other scaffold medium) may partially or completely fill the microfluidic channel, and cells may be cultured on or in the hydrogel. In addition, in some embodiments, there may be one or more additional microfluidic channels. These may be used for various purposes, e.g., to deliver fluids such as cell media, provide nutrients, remove waste, or the like, to or from the hydrogel. Such channels may be free of hydrogel in certain embodiments. In addition, in some cases such as those discussed below, no physical barrier may be present between the hydrogel and fluid that may be present within the second microfluidic channel.

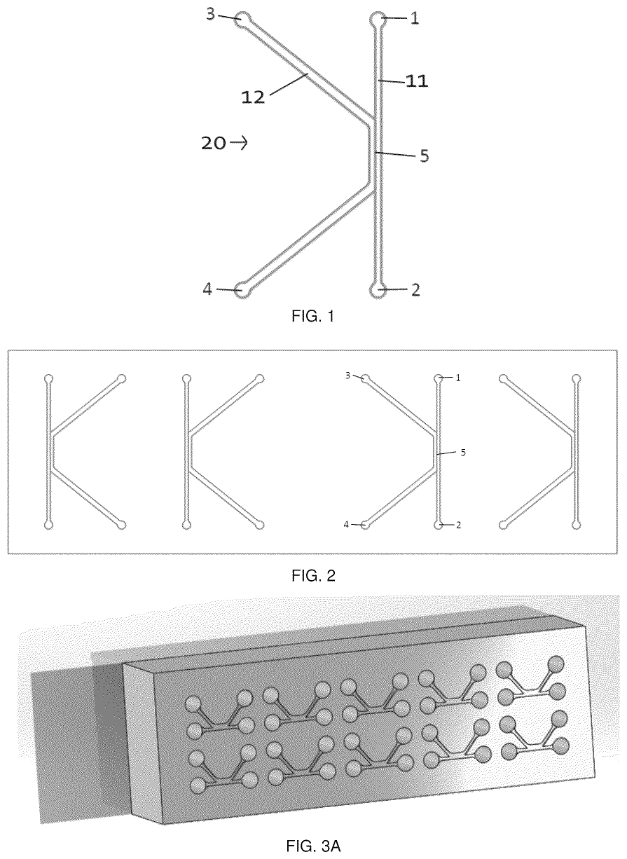

One non-limiting example of such a microfluidic device is shown in FIG. 1 with sample device 20. In this figure, first microfluidic channel 11 connects inlet 1 to outlet 2, while second microfluidic channel 12 connects inlet 3 to outlet 4. First microfluidic channel 11 may be filled with a hydrogel or another scaffold medium, while second microfluidic channel 12 may be empty, e.g., such that during use of the microfluidic device, a fluid (e.g., cell media) can flow from inlet 3 to outlet 4 (or vice versa in some cases). This may be used, for example, to perfuse the cells within the microfluidic device, for example, contained on or within the hydrogel within first microfluidic channel 11.

Also shown in this figure is common interconnect region 5, in which first microfluidic channel 11 and second microfluidic channel 12 come into fluidic contact with each other, e.g., such that a fluid could flow from one channel to the other if both channels were empty. In some cases, both channels may be positioned to be parallel to each other within common interconnect region 5, and in some cases, no physical barrier may be present within common interconnect region 5 that partially or completely separates first microfluidic channel 11 and second microfluidic channel 12 from each other. For example, no pillars, columns, bumps, phaseguides, ridges, or other barriers may be present that separates first microfluidic channel 11 and second microfluidic channel 12.

In some embodiments, one or more microfluidic channels within a microfluidic device may be treated to render them more hydrophilic. This may be particularly useful, for instance, for certain polymers that are relatively hydrophobic that may be used in the microfluidic device. For example, in one set of embodiments, a microfluidic channel may be at least partially defined using polymers such as polystyrene (PS), polycarbonate (PC) or polymethylmethacrylate (PMMA), which are known to be relatively hydrophobic. Due to their hydrophobicity, it can be difficult to pass aqueous fluids through such microfluidic channels. Accordingly, as discussed herein, one or more walls of such microfluidic channels may be at least partially coated with a suitable polymer that may be more hydrophilic. Non-limiting examples of such polymers include polyvinylpyrrolidone (PVP), poly(ethylene glycol) (PEG), poly(vinyl alcohol) (PVA), or the like. Additional techniques for producing such devices are discussed in a US provisional patent application, filed on Sep. 30, 2022, entitled “Methods and Systems for Functionalizing Surfaces for Microfluidic Devices or Other Applications,” U.S. Ser. No. 63/412,273, incorporated herein by reference in its entirety.

In some embodiments, a fluid may be passed through microfluidic channels within the device. In some cases, such a fluid may contain a precursor of a hydrogel or other scaffold medium, which may be treated (e.g., hardened) to form a hydrogel or other scaffold medium. In some cases, the hydrogel or other scaffold medium may be formed on the polymer or other coating material within the microfluidic channel, which may be more hydrophilic and allow the fluid to contact and readily flow through the microfluidic channel. Thus, certain embodiments such as discussed herein are generally directed to microfluidic channels having a polymer or other coating material, and a hydrogel that is in contact with it, e.g., such that the polymer is positioned between the hydrogel (or other scaffold medium) and one or more walls of the microfluidic channel.

In addition, in certain embodiments, the hydrogel (or other coating material) may be substantively contained within a microfluidic channel, e.g., within a common interconnect region having other microfluidic channels, for example, without the hydrogel being blocked due to pillars, columns, bumps, phaseguides, ridges or other physical barriers, e.g., as discussed in a US provisional patent application, filed on Sep. 30, 2022, entitled “Techniques and Systems for Creating Spatially Controlled Fluidic Flows in Surface Functionalized Microfluidic Devices,” U.S. Ser. No. 63/412,279, incorporated herein by reference in its entirety.

Accordingly, certain embodiments such as discussed herein are generally directed to microfluidic channels having a polymer or other coating material, and a hydrogel or other scaffold medium in contact with the polymer or other coating material. Optionally, cells may be grown or cultured on or in the hydrogel or other scaffold medium, e.g., as discussed herein, e.g., to emulate various functions of specific organs, such as in an organ-on-a-chip device, and such cells can be studied, e.g., using techniques such as imaging, analysis of media exiting the microfluidic device after being exposed to the cells, or the like.

The above discussion is a non-limiting example of certain embodiments that are generally directed microfluidic devices that can contain cells, e.g., in contact with a hydrogel or another scaffold medium. However, other embodiments are also possible. Accordingly, more generally, various aspects as discussed herein are directed to various systems and methods directed to microfluidic devices, e.g., containing hydrogels in certain embodiments.

One aspect, for example, is generally directed to a microfluidic device, e.g., having one or more microfluidic channels defined in a substrate. The substrate may have any suitable shape or configuration, including square, rectangular, circular, etc. In some cases, the substrate may include one or more layers of material. In certain cases, one or more layers of the substrate may be formed out of materials such as pressure-sensitive adhesives, or other materials, including any of those described herein. For instance, the microfluidic device may include one, two, three, four, or more layers, and one or more of the layers may contain or define one or more microfluidic channels therein. In addition, in some cases, larger channels, tubes, chambers, reservoirs, fluidic pathways, etc. may also be defined within a substrate, e.g., using one or more layers.

In addition, in one set of embodiments, a coating material may be present on one or more walls defining a microfluidic channel, for example, to alter the hydrophilicity of the walls. For example, the coating material may increase or decrease the hydrophilicity of at least one of the walls defining a microfluidic channel. Different walls of the microfluidic channel may independently have the same or different hydrophilicities, for example, by coating different walls with different coating materials (or no coating material). Without wishing to be bound by any theory, it should be understood that, due to the small and cramped nature of the microfluidic channels, a fluid within a microfluidic channel may interact with the walls of the microfluidic channels, which can affect the flow properties of the fluid flowing through the channel. Thus, in some embodiments, the hydrophilicities of the walls forming a microfluidic channel may affect the flow of fluid through the channel.

For example, in one set of embodiments, a fluid containing a polymer or other suitable coating material may be flowed through a microfluidic channel, and in some cases, the fluid may be constrained to prevent it from entering other microfluidic channels. For instance, in some cases, a fluid may enter a first microfluidic channel in a common interconnect region, but due to the presence of adhesive or other feature that masks other microfluidic channels within the common interconnect region, the fluid is not able to enter the masked channels. In some cases, the coating material may be deposited onto one or more walls containing the fluid. This may be useful, for example, for altering the hydrophilicity of the walls, for creating a surface for adhering other materials to the walls, for altering the opacity of the walls, or other applications. In addition, other methods of adding a coating material may be used, for example, dip coating or drop casting.

Non-limiting examples of polymers that may be deposited onto one or more walls of a microfluidic channel, e.g., to form a coating thereon, include polyvinylpyrrolidone (PVP), poly(ethylene glycol) (PEG), poly(vinyl alcohol) (PVA), polylysine, or the like. In addition, in some cases, the coating materials may include other materials, in addition to or instead of polymers such as these, for example, ECM attachment factor. In some cases, coating materials, including polymers such as these, may be used to alter or increase the hydrophilicity of the microfluidic channel. In some cases, the increased hydrophilicity may be determined as a change in water contact angle, or by applying 2 microliters of water to a surface of the hydrophilic coating, and measuring a spread of water onto the surface of at least 10 mm².

In one set of embodiments, a hydrogel or other scaffold medium may be positioned on, adjacent to, or attached to the coating, e.g., such that the coating is positioned or located between the hydrogel and a wall of the microfluidic channel. The hydrogel (or other scaffold medium) may be applied, for example, by flowing a fluid containing a hydrogel or other scaffold medium precursor through a microfluidic channel, and treating the precursor to form the hydrogel or other scaffold medium. For example, the hydrogel precursor may be caused to harden to form a hydrogel. In some cases, the fluid containing the precursor may be a hydrophilic fluid, such as water, saline, or buffer, and in certain embodiments, the fluid may be preferentially attracted to a hydrophilic coating material, e.g., that may be present on one or more walls of a microfluidic channel. Examples of hydrophilic coatings include any of those described herein. In some cases, the fluid containing the precursor may preferentially be contained within a first microfluidic channel (e.g., within a common interconnect region as describe herein), without entering other microfluidic channels. Upon treatment (e.g., hardening), the resultant hydrogel (or other scaffold medium) may be positioned on the coating material within the first microfluidic channel, while other microfluidic channels may be substantially free of the hydrogel or other scaffold medium.

Non-limiting examples of hydrogels (e.g., that can be used as an extracellular matrix for cells) include collagen (e.g., Type I collagen, Type II collagen, Type III collagen, etc.), Matrigel®, methacrylated gelatin (Gel-MA), fibrin, alginate, hyaluronic acid, polyacrylamide, poly(ethylene glycol), poly(vinyl alcohol), agarose, agar, chitosan, poly(RADARADARADARADA) (PuraMatrix), poly(AEAEAKAKAEAEAKAK) (EAK16), poly(KLDLKLDLKLDL) (KLD12), or the like. In addition, more than one of these and/or other materials may be present in a hydrogel in certain instances. The collagen may arise from any suitable source, e.g., bovine collagen, rat collagen, fish (marine) collagen, chicken collagen, porcine collagen, sheep collagen, or the like. Other hydrogels will be known by those of ordinary skill in the art. In some embodiments, hydrogels such as these can be formed by flowing a fluid containing a hydrogel precursor, and causing the precursor to form the hydrogel, for example, using a change in temperature (e.g., cooling the device), exposure to ultraviolet radiation, exposure to a chemical, or the like.

In addition, other scaffold media can be used in certain embodiments, e.g., instead of or in addition to a hydrogel as discussed herein. Thus, it should be understood that hydrogels are described herein by way of example only. Non-limiting examples of other scaffold media that may be used in certain embodiments include paraffin, waxes, or the like. These may be added, for example, by flowing a fluid containing an scaffold medium precursor into a microfluidic channel within the device, and treating the precursor to form the scaffold medium within the device. For example, a paraffin or a wax may be introduced into a device at a temperature where the material is liquid, and treated (e.g., cooled) to solidify the medium within the microfluidic device.

In addition, in one set of embodiments, the scaffold medium may be substantially transparent, e.g., to allow for imaging of cells, such as is described herein. As a non-limiting example, in one embodiment, a hydrogel comprising collagen may be used.

According to one set of embodiments, the hydrogel or other scaffold medium may be exposed to cells, which may be grown or cultured on or in the hydrogel or other scaffold medium in some embodiments. Any suitable technique may be used to apply the cells. In some cases, for instance, the cells may be suspended in solution, which is flowed past the hydrogel or other scaffold medium, e.g., within the common interconnect region, and allowed to incubate there to promote attachment of the cells. In some cases, this process may occur over a period of at least 24 hours, or other suitable times. In addition, in some cases, the cells may be mixed with a fluid containing a hydrogel precursor or other scaffold medium precursor, e.g., prior to introduction to the microfluidic device. The cells may then be incubated and allowed to become embedded within the hydrogel or other scaffold medium. Those of ordinary skill in the art will be familiar with techniques for attaching cells to a suitable scaffold medium. Without wishing to be bound by any theory, it is believed that culturing cells on or in such an scaffold medium, e.g., a hydrogel, may more closely approximate the conditions that the cells naturally grow in, e.g., as opposed to a 2-dimensional surface. Accordingly, such cells may respond more similarly and appropriately when cultured in a 3-dimensional environment, such as a hydrogel.

Examples of cells that may be cultured on or in a hydrogel or other scaffold medium include, but are not limited to, mammalian cells such as human cells. Specific non-limiting examples include fibroblasts, lung cells, liver cells, fat cells, kidney cells, intestinal cells, brain cells, epithelial cells, endothelial cells, stromal cells, immune cells, or the like. In some cases, the cells may be stem cells, such as pluripotent stem cells, totipotent stem cells, multipotent stem cells, etc. Other cell types are also possible. In some cases, more than one type of cell may be present, e.g., liver cells and fibroblasts. In addition, in certain embodiments, the cells may produce organoids, tubes, or other 3-dimensional structures, e.g., depending on the cells being cultured.

In some cases, the cells may be cultured within the microfluidic device, for example, within a common interconnect region. In some cases, for instance, in a common interconnect region, a first microfluidic channel may contain a hydrogel or other scaffold medium, and cells that are in contact with the hydrogel or other scaffold medium. The common interconnect region may also comprise a second microfluidic channel that can contain a fluid (for example, cell media) that is able to maintain the cells within the hydrogel. Non-limiting examples of cell media include MEM, DMEM, RPMI, IMDM, F-10, or the like. Those of ordinary skill in the art will be able to select appropriate cell media, e.g., based on the type of cells that are present within the common interconnect region. In some cases, fluid is able to flow in and out of the common interconnect region, e.g., as the hydrogel (or other scaffold medium) may only partially fill the common interconnect region, thereby allowing fluid flow to occur through the common interconnect region. In addition, in some cases, the fluid may be in direct contact with the hydrogel or other scaffold medium, e.g., without having to circumvent a pillar, column, or other physical barrier. Thus, in some embodiments, there may be a barrierless interface between the hydrogel or other scaffold medium and a fluid (e.g., cell media) within the common interconnect region. This may allow the cells to be perfused by the cell media, e.g., to provide nutrients or dissolved gases, remove waste, or the like.

The microfluidic channels within the microfluidic device may have any configuration within the device, and there may be one or more than one such channel, which may independently be the same or different. A microfluidic channel may have any cross-sectional shape (circular, oval, triangular, irregular, square or rectangular, or the like) and can be covered or uncovered. The microfluidic channels may be used to move or process fluid within the substrate in any of a number of ways, for example, to allow fluids to flow from one or more inlets, through the microfluidic channel, to one or more outlets.

In some cases, a microfluidic channel may have a maximum cross-sectional dimension of less than 10 mm, less than 8 mm, less than 7 mm, less than 6 mm, less than 5 mm, less than 3 mm, less than 2 mm, and in certain cases, less than 1 mm, less than 500 micrometers, less than 300 micrometers, less than 200 micrometers, less than 100 micrometers, less than 50 micrometers, less than 30 micrometers, less than 20 micrometers, less than 10 micrometers, less than 5 micrometers, etc. In addition, a microfluidic channel may have a maximum cross-sectional dimension of at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 6 mm, at least 7 mm, at least 8 mm, at least 10 mm, etc. Any combination of these is also possible. For instance, a microfluidic channel may have a maximum cross-sectional dimension of between 10 micrometers and 30 micrometers, between 100 micrometers and 500 micrometers, between 300 micrometers and 1 mm, or the like.

In some cases, all of the channels within a substrate or a layer may be microfluidic channels. However, in other cases, larger channels, tubes, chambers, reservoirs, fluidic pathways, etc. may also be present. Those of ordinary skill in the art will be familiar with microfluidic channels and systems and methods of making substrates containing microfluidic channels (and/or other channels).

In one set of embodiments, the microfluidic channels may have any suitable configuration. If more than one microfluidic channel is present, the channels may independently have the same or different lengths. In some cases, one or more microfluidic channels may intersect, for example, in a T, Y, or a + intersection, or within a common interconnect region such as described herein, etc. Other types of intersections are also possible. A microfluidic channel, in some cases, may be substantially straight between an inlet and an outlet. In addition, in some cases, a microfluidic channel may have one, two, or more bends, curves, or the like between an inlet and an outlet. (As a non-limiting example, as is shown in FIG. 1, microfluidic channel 12 has two bends between inlet 3 and outlet 4.) If more than one microfluidic channel is present, the microfluidic channels may independently have the same or different configurations. In some cases, there may be 0, 1, 2, or more intersections with other microfluidic channels between an inlet and an outlet of the microfluidic channel. In addition, in one set of embodiments, a microfluidic channel may pass between a single port and a microfluidic interconnect region, e.g., there may not necessarily be both an inlet and an outlet of a microfluidic channel.

A microfluidic channel may have any suitable pathlength, e.g., length along the channel as a fluid flows between an inlet and an outlet of the channel. If more than one microfluidic channel is present, the microfluidic channels may independently have the same or different pathlengths. For instance, in some embodiments, a microfluidic channel may have a pathlength of at least 1 mm, at least 2 mm, at least 3 mm, at least 4 mm, at least 5 mm, at least 6 mm, at least 7 mm, at least 8 mm, at least 9 mm, at least 10 mm, at least 12 mm, at least 15 mm, at least 20 mm, etc. In some embodiments, the maximum pathlength may no more than 20 mm, no more than 15 mm, no more than 12 mm, no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of a microfluidic channel may be between 5 mm and 7 mm, between 4 mm and 8 mm, between 2 mm and 6 mm, etc.

In one set of embodiments, two, three, four, five, or more microfluidic channels may meet at a common interconnect region. In some cases, some or all of the microfluidic channels may be positioned to be parallel to each other within the common interconnect region, and in some cases, no physical barrier (e.g., pillars, columns, bumps, phaseguides, ridges, etc.) may be present within the common interconnect region that partially or completely separates the microfluidic channels from each other. Thus, for example, a fluid could flow from one channel within the common interconnect region to another channel within the common interconnect region if both channels were empty.

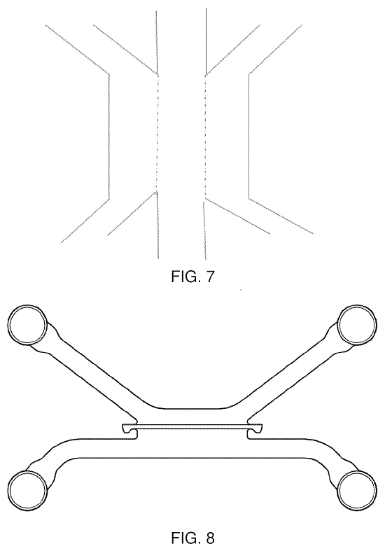

Non-limiting examples of a common interconnect region with two microfluidic channels are shown in FIGS. 1 and 8, while a non-limiting example of a common interconnect region with three microfluidic channels is shown in FIG. 7. The common interconnect region in some cases, may be treated as a microfluidic channel portion that is composed of two or more microfluidic channels that are in fluidic contact with each other and are generally positioned parallel to each other within the region, although the microfluidic channels may not necessarily be parallel outside of the common interconnect region. In a common interconnect region, the channels are not separated (e.g., by physical barriers such as pillars, columns, bumps, phaseguides, ridges, etc.), and the microfluidic channels can come into contact with each other such that the microfluidic channels in fluidic contact, e.g., to allow fluid flow between channels to occur within the common interconnect region. For example, a first microfluidic channel may have a first inlet and a first outlet, and a second microfluidic channel may have a second inlet and a second outlet, and the first and second microfluidic channels may come into contact and be positioned parallel to each other within the common interconnect region between their respective inlets and outlets (although outside of the common interconnect region, they may or may not also be parallel).

As a non-limiting example, as discussed herein, a first microfluidic channel may contain a hydrogel or other scaffold medium, while a second microfluidic channel may contain a fluid (e.g., cell media), and within the common interconnect region, the fluid is able to come into direct contact with the hydrogel or other scaffold medium, e.g., without having to circumvent a physical barrier, such as a pillar or a column. Accordingly, in certain embodiments, there may be a barrierless interface in a common interconnect region between a first fluid or medium in a first microfluidic channel (for example, a hydrogel or other scaffold medium), and a second fluid or medium in a second microfluidic channel (for example, cell media). For instance, in some embodiments, no interface material or physical barrier separating the first fluid or medium from the second fluid or medium may be present. Thus, for example, a hydrogel or other scaffold medium may partially fill the common interconnect region, for example, such that at least 20%, at least 30%, at least 40%, at least 50%, at least 60%, at least 70%, at least 80%, and/or no more than 80%, no more than 70%, no more than 60%, no more than 50%, no more than 40%, no more than 30%, or no more than 20% of any cross-section of the common interconnect region is not filled with the hydrogel or other scaffold medium. In some embodiments, the hydrogel (or other scaffold medium) partially fills the common interconnect region such that the hydrogel does not prevent bulk fluid flow through at least a portion of the common interconnect region.

In some cases, at least a portion, or all, of the common interconnect region may be substantially straight. In addition, in certain embodiments, the microfluidic channels are positioned within the common interconnect region to be substantially parallel to each other. The parallel microfluidic channels can be used to define an imaginary channel axis that passes through the common interconnect region, e.g., in a direction defined by the direction that the parallel microfluidic channels are oriented. However, in certain cases, one or more of the microfluidic channels may be at an angle relative to other microfluidic channels within the common interconnect region.

In some embodiments, the common interconnect region may have a longest dimension along the channel axis (if present) of at least 1 mm, at least 2 mm, at least 3 mm, at least 4 mm, at least 5 mm, at least 6 mm, at least 7 mm, at least 8 mm, at least 9 mm, at least 10 mm, etc. In addition, the common interconnect region may have a longest dimension along the channel axis of no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the common interconnect region may have a longest dimension of between 5 mm and 7 mm, between 4 mm and 8 mm, between 2 mm and 6 mm, etc.

In certain embodiments, the common interconnect region may have a maximum cross-sectional dimension, or a maximum dimension orthogonal to the channel axis (if present), of at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, at least 100 mm, etc. In addition, in certain embodiments, the common interconnect region may have maximum dimensions of no more than 100 mm, no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, etc. In addition, combinations of any of these are also possible. For example, a common interconnect region may have maximum dimensions of between 100 micrometers and 300 micrometers, between 5 mm and 10 mm, between 500 micrometers and 2 mm, or the like.

In one set of embodiments, two or more microfluidic channels within a common interconnect region may be separated using a trench, e.g., on or in a wall of the common interconnect region. One non-limiting example of such a trench is shown in FIG. 8.

More than one trench may also be present in some cases, e.g., on opposed surfaces within the common interconnect region. Without wishing to be bound by any theory, it is believed that a fluid flowing in a channel may be attracted to a channel surface, e.g., due to similar hydrophilicities (e.g., if both are relatively hydrophilic or hydrophobic) and/or capillary action, which may facilitate the flow of the fluid within the channel. However, it may be difficult in certain embodiments for such a fluid to be able to cross a trench, e.g., if the volume of fluid is not too great. For example, the trench may exhibit a different hydrophilicity (e.g., one that does not promote attraction with the fluid), and/or the shape of the trench may discourage the fluid from being able to cross, e.g., due to the dimensions of the trench. In some embodiments, the trench may facilitate the flow of fluid through one channel within the common interconnect region, for example, without the fluid flowing into another channel within the common interconnect region. In addition, in certain embodiments, the trench may be treated, e.g., as discussed herein, to render it more hydrophilic or hydrophobic. For example, a coating material, such as a hydrophobic polymer, may be coated on at least a portion of the trench.

Accordingly, in some embodiments, a trench may be positioned within a common interconnect region between a first microfluidic channel and a second microfluidic channel. The trench may run along the length of the common interconnect region in some embodiments, e.g., to separate the two channels. Such a trench may thus provide physical separation of the channels, e.g., without the use of physical barriers (e.g., pillars, columns, bumps, phaseguides, ridges, etc.) to separate the channels. Such trenches are also discussed in more detail in a US provisional patent application, filed on Sep. 30, 2022, entitled “Techniques and Systems for Creating Spatially Controlled Fluidic Flows in Surface Functionalized Microfluidic Devices,” U.S. Ser. No. 63/412,279, incorporated herein by reference in its entirety. However, it should be understood that in other embodiments, a trench may be used in conjunction with pillars, columns, bumps, phaseguides, ridges, or other barriers.

The trench may have any suitable dimensions or shape within the common interconnect region. For example, the trench may be substantially straight, or the trench may be bent or curved in certain embodiments. In some cases, the trench may have a length comparable to the length of the common interconnect region. In some embodiments, the trench may have a maximum length of at least 1 mm, at least 2 mm, at least 3 mm, at least 4 mm, at least 5 mm, at least 6 mm, at least 7 mm, at least 8 mm, at least 9 mm, at least 10 mm, etc. In some embodiments, the maximum length may no more than 10 mm, no more than 9 mm, no more than 8 mm, no more than 7 mm, no more than 6 mm, no more than 5 mm, no more than 4 mm, no more than 3 mm, no more than 2 mm, no more than 1 mm, etc. Combinations of these are also possible in other embodiments. For example, the length of the trench may be between 5 mm and 7 mm, between 4 mm and 8 mm, between 2 mm and 6 mm, etc.

In some embodiments, a trench may have a cross-sectional dimension of at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, etc. In addition, in some embodiments, the trench may have a cross-sectional dimension of no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, etc. In addition, combinations of any of these are also possible, e.g., a trench may have a cross-sectional dimension of between 100 micrometers and 300 micrometers, between 200 micrometers and 1 mm, between 500 micrometers and 3 mm, etc. The trench may have a constant cross- sectional dimension, or a cross-sectional dimension that varies in some embodiments.

In addition, the trench may have any suitable depth. The depth may be independent of the cross-sectional dimension. In some embodiments, the depth may be at least 1 micrometer, at least 2 micrometers, at least 3 micrometers, at least 5 micrometers, at least 10 micrometers, at least 20 micrometers, at least 30 micrometers, at least 50 micrometers, at least 100 micrometers, at least 200 micrometers, at least 300 micrometers, at least 500 micrometers, at least 1 mm, at least 2 mm, at least 3 mm, at least 5 mm, at least 10 mm, at least 20 mm, at least 30 mm, at least 50 mm, etc. In addition, in some cases, the depth may be no more than 50 mm, no more than 30 mm, no more than 20 mm, no more than 10 mm, no more than 5 mm, no more than 3 mm, no more than 2 mm, no more than 1 mm, no more than 500 micrometers, no more than 300 micrometers, no more than 200 micrometers, no more than 100 micrometers, no more than 50 micrometers, no more than 30 micrometers, no more than 20 micrometers, no more than 10 micrometers, no more than 5 micrometers, no more than 3 micrometers, no more than 2 micrometers, no more than 1 micrometer, etc. In addition, combinations of any of these are also possible in certain embodiments. For instance, the trench may have a depth of between 2 mm and 3 mm, between 1 mm and 10 mm, between 100 micrometers and 2 mm, etc. The trench may have a constant depth, or a depth that varies in some cases.

In addition, according to one set of embodiments, a first microfluidic channel and a second microfluidic channel may meet at a common interconnect region where the channels are positioned parallel within the common interconnect region. As previously discussed, there may optionally be a trench positioned between the first microfluidic channel and the second microfluidic channel at the common interconnect region. In some cases, the first microfluidic channel may be a straight channel between a first inlet and an outlet, while the second microfluidic channel may include bends on either side of the common interconnect region between a second inlet and a second outlet, thereby forming a K-shaped structure. A non-limiting example of such a structure can be seen in FIG. 1. In some cases, as discussed herein, one or more of the channels may contain a hydrogel or other scaffold medium, e.g., such that the hydrogel or other scaffold medium does not completely fill the common interconnect region and a fluid can pass between an inlet and an outlet through a microfluidic channel within the common interconnect region, e.g., in a microfluidic channel that is free of the hydrogel or other scaffold medium.

In one set of embodiments, there may be a plurality of repeat units on a substrate, e.g., repeat units including one or more microfluidic channels or common interconnect regions, such as those described herein. For instance, there may be at least 3, at least 5, at least 10, at least 20, at least 30, at least 40, at least 50, at least 60, at least 70, at least 80, at least 90, at least 100, etc. repeat units on a substrate. The repeat units may be all identically oriented, or they may be differently oriented (e.g., rotated, flipped, etc.) in certain embodiments. In addition, in some cases, two, three, or more types of repeat units may be present on a substrate, e.g., having dissimilar configurations.

In some embodiments, the repeat units may be regularly arranged on a substrate. For instance, the repeat units may be arranged as a square, a rectangle, a circle, a hexagonal configuration, or the like. In addition, the repeat units may be irregularly arranged in certain cases. As an example, the repeat units may be arranged in a 2×n configuration, where n is 1, 2, 3, 4, 5, 6, 7, 8, 9, 10, or the like. As another non-limiting example, the repeat units may be arranged in a 3×n configuration, a 4×n configuration, a 6×n configuration, an 8×n configuration, a 12×n configuration, a 16×n configuration, or the like. For example, the repeat units may be arranged in a 6×6 configuration, an 8×8 configuration, or the like, a 16×16 configuration, or the like.

The microfluidic channels, according to one set of embodiments, may be contained with a substrate having dimensions comparable to a microscope slide, e.g., arranged into a plurality of repeat units on the substrate. For example, the substrate may have dimensions of 75 mm×25 mm, 75 mm×26 mm, 46 mm×28 mm, 46 mm×27 mm, 75 mm×38 mm, 76 mm×51 mm, 76 mm×52 mm, etc. In some cases, such dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment able to handle microscope slides.

In another set of embodiments, the microfluidic channels may be contained with a substrate having dimensions comparable to a microwell plate, e.g., one having ANSI dimensions of 128 mm×85 mm, e.g., arranged into a plurality of repeat units on the substrate. In some cases, the dimensions may vary somewhat (for example, by +/−1 mm, +/−2 mm, or +/−5 mm, etc.), e.g., to allow for manufacturing tolerances or the like. Such dimensions may be useful in some embodiments, e.g., to interface with laboratory equipment, such as plate readers or liquid handling robots that are able to handle microwell plates. In addition, in some embodiments, one or more inlets and/or outlets may be positioned within the substrate to match the locations of wells on a microwell plate, e.g., the center locations of the wells on a 24-well standard microplate, a 48-well standard microplate, a 96-well standard microplate, a 384-well standard microplate, or a 1536-well standard microplate, etc.

The substrate may be formed from any suitable materials. In some cases, the substrate may be formed from one, two, three, four, five, or more layers of materials, which may independently be the same or different. For instance, a layer within the substrate may comprise glass or a polymer. Non-limiting examples of polymers include polystyrene, polycarbonate, polymethylmethacrylate, polycarbonate, polypropylene, cyclic olefin polymers (COP), cyclic olefin copolymers (COC), polyethylene terephthalate (PET), or the like. For example, an outer or end layer of the substrate may comprise glass or polymer, which may be useful for protecting internal components of the microfluidic device. In addition, as discussed herein, one or more of the layers of the microfluidic channel may be chosen to be substantially transparent.

In addition, in one set of embodiments, the substrate may include a layer comprising a pressure-sensitive adhesive (PSA). In some cases, a layer may be formed from a PSA. Non-limiting examples of pressure-sensitive adhesives include acrylic-based adhesives, silicone-based adhesives (e.g., polydimethylsiloxane), polyurethane-based adhesives, or the like. Certain PSAs may be readily obtainable commercially. In some embodiments, pressure-sensitive adhesives may be particularly useful for defining one or more features, such as microfluidic channels or other channels, tubes, chambers, reservoirs, fluidic pathways, trenches, or the like, e.g., as discussed herein. In some embodiments, for example, one or more features may be defined within a pressure-sensitive adhesive layer, e.g., using cutting techniques such as laser cutting, die cutting, or the like. In some cases, such features may be removed from the pressure-sensitive adhesive, thereby defining the feature within the pressure-sensitive adhesive. In addition, in some cases, such pressure-sensitive adhesives may be pressed or adhered onto another layer, e.g., to form a microfluidic device. For example, in one set of embodiments, a pressure-sensitive adhesive layer may be sandwiched between two other layers (which may be compositionally the same, or different). In addition, in some embodiments, more than one pressure-sensitive adhesive may be present within a microfluidic device.

In some cases, the substrate, or one or more layers, may be chosen to be substantially transparent, for example, to allow for imaging of the common interconnect region (for example, cells within the common interconnect region), or other locations within the substrate. In some embodiments, the entire substrate may be substantially transparent. A variety of techniques may be used for imaging, including light or optical microscopy, confocal microscopy, fluorescence microscopy, microwell plate readers, or the like. Those of ordinary skill in the art will be aware of other suitable imaging techniques. In some cases, multiple locations within a microfluidic device may be studied, e.g., sequentially and/or simultaneously. For example, in some embodiments, the microfluidic device may contain a plurality of repeat units that can be independently determined. In certain embodiments, fluid (e.g., cell media) may be flowed through a common interconnect region (e.g., to perfuse cells, etc., as discussed herein) during imaging (for example, uni- or bidirectionally), although in other cases no such flow may occur during imaging.

In one set of embodiments, microfluidic devices such as those described herein may be used for the study of cells or other constructs, such as organoids, tubes, or other 3-dimensional structures. These may be present, for example, in a common interconnect region, such as is described herein. In some cases, for example, the cells may act as an organ, e.g., the cells may be able to emulate one or more functions of a specific organ. In some embodiments, microfluidic devices having such cells or other constructs may be used to study their function, for example, microscopically (e.g., using imaging such as discussed herein), and/or by analyzing media exiting the microfluidic device (e.g., after being exposed to the cells or other constructs), etc. For example, fluid (e.g., cell media) exiting the microfluidic device may be studied to determine proteins, enzymes, nucleic acids, nutrients, waste gases, or the like, e.g., after exposure to the cells or other constructs.

In addition, in some cases, microfluidic devices having such cells or other constructs may be used to determine the effects of agents thereon. For example, cells or other constructs contained within a microfluidic device (e.g., in a common interconnect region) may be exposed to one or more agents that are suspected of being able to interact, and in some cases alter, such cells or other constructs. The agent may be, for example, a pharmaceutical, a drug, a toxin, a biomolecule, or the like. The agent may be supplied to the cells or other constructs, e.g., separately, or along with cell media that is introduced to the microfluidic device. One or more agents may be used.

In addition, in some cases, as discussed, a microfluidic device may contain more than one such system, e.g., as in a plurality of repeat units on a substrate. In some cases, multiple experiments may be performed simultaneously, e.g., exposure to different agents, and/or the same agents at different concentrations, control experiments, etc., may be performed using different repeat units within the microfluidic device. These experiments may be arranged, e.g., systematically or randomly within the microfluidic device.

In addition, certain aspects are generally directed to methods of making microfluidic devices such as those described herein. Additional techniques for making microfluidic devices include those described in a US provisional patent application, filed on Sep. 30, 2022, entitled “Methods and Systems for Functionalizing Surfaces for Microfluidic Devices or Other Applications,” U.S. Ser. No. 63/412,273 incorporated herein by reference.

For example, in one set of embodiments, a microfluidic device may be formed from a first layer (e.g., a relatively hydrophobic polymer such as polystyrene) and a second layer (e.g., a pressure-sensitive adhesive). In some cases, the second layer may be pressed onto the first layer to form a substrate. In some cases, the second layer may be pre-cut (e.g., laser-cut) with one or more microfluidic channels, or other suitable channels, chambers or fluidic pathways, etc. After adhesion, at least a portion of the second layer may be removed, e.g., to define a suitable channel or other fluidic pathways.

In some cases, the exposed portions of the first layer and/or the second layer may be treated with a polymer or other coating material, e.g., to render them more hydrophilic. Thus, for example one or more walls defining a microfluidic channel may be partially or fully coated with a polymer or other coating material. In addition, in some cases, before a coating material is added, one or more of the surfaces (e.g., of the microfluidic channel) may be treated to facilitate the addition of the coating material. Non-limiting examples of suitable surface treatments include oxygen plasma treatment, corona plasma treatment, or the like.

The polymer or other coating material may be added to the exposed portions using any suitable technique. Examples of suitable polymers include PVP, PEG, PVA, or other polymers such as those described herein. For example, a fluid containing the polymer (or other coating material) may be added to the exposed portions, e.g., by flowing from an inlet to an outlet of a microfluidic channel, and the polymer may be able to coat the exposed surfaces (for example, portions of the surface that had been surface treated as discussed above). In some cases, after waiting for a suitable period of time, the fluid containing the polymer may also be removed, thereby resulting in coated portions within the microfluidic channels.

In some cases, after treatment, other portions (e.g., portions that may have been pre-cut with one or more microfluidic channels, or other suitable channels, chambers or fluidic pathways, etc.) may be removed from the second layer, thereby resulting in a microfluidic device having channels with different hydrophilicities.

In one set of embodiments, optionally, an additional, third layer may be added on top to close the microfluidic channels, e.g., to produce the final microfluidic device. The third layer may, for example, be a polymer layer, and it may be the same or different from the first layer of the device. In some cases, the third layer may include one or more ports or holes to define inlets and/or outlets, for example, to allow fluids to flow into and/or out of the device, e.g., through one or more microfluidic channels.

In some embodiments, a fluid may be passed through microfluidic channels within the device. In some cases, such a fluid may contain a precursor of a hydrogel or other scaffold medium, which may be treated (e.g., hardened) to form a hydrogel or other scaffold medium. In some cases, the hydrogel or other scaffold medium may be formed on the polymer or other coating material within a microfluidic channel, which may be more hydrophilic and allow the fluid to contact and readily flow through the microfluidic channel. Thus, certain embodiments such as discussed herein are generally directed to microfluidic channels having a polymer or other coating material, and a hydrogel that is in contact with it, e.g., such that the polymer is positioned between the hydrogel (or other scaffold medium) and one or more walls of the microfluidic channel.

In addition in certain embodiments, the hydrogel (or other coating material) may be substantively contained within a microfluidic channel, e.g., within a common interconnect region having other microfluidic channels, for example, without the hydrogel being blocked due to pillars, columns, bumps, phaseguides, ridges, or other physical barriers. However, it should be understood that in other embodiments, a hydrogel (or other coating material) may be used in conjunction with pillars, columns, bumps, phaseguides, ridges, or other barriers.

The following are each incorporated herein by reference in their entireties: U.S. Provisional Patent Application Ser. No. 63/412,174, filed Sep. 30, 2022, entitled “Microfluidic Devices Containing Hydrogels, and Techniques for Making and Using”; U.S. Provisional Patent Application Ser. No. 63/412,273, filed Sep. 30, 2022, entitled “Methods and Systems for Functionalizing Surfaces for Microfluidic Devices or Other Applications”; U.S. Provisional Patent Application Ser. No. 63/412,279, filed Sep. 30, 2022, entitled “Techniques and Systems for Creating Spatially Controlled Fluidic Flows in Surface Functionalized Microfluidic Devices”; U.S. Provisional Patent Application Ser. No. 63/437,954, filed Jan. 9, 2023, entitled “Edge Effect Systems and Methods for Functionalized Microfluidic Devices”; and U.S. Provisional Patent Application Ser. No. 63/437,955, filed Jan. 9, 2023, entitled “Pipette Interface Systems and Methods for Viscous Fluid Injection.”

In addition, the following patent applications, filed on even date herewith, are incorporated herein by reference in their entireties: a PCT application entitled “Methods and Systems for Functionalizing Surfaces for Microfluidic Devices or Other Applications”; a PCT application entitled “Techniques and Systems for Creating Spatially Controlled Fluidic Flows in Surface Functionalized Microfluidic Devices”; a PCT application entitled “Edge Effect Systems and Methods for Functionalized Microfluidic Devices”; and a PCT application entitled “Pipette Interface Systems and Methods for Viscous Fluid Injection.”

Furthermore, the following patent applications, filed on even date herewith, are incorporated herein by reference in their entireties: a US design application entitled “Fluid Channel”; a US design application entitled “Fluid Channel Trench”; a US design application entitled “Well Plate”; a US design application entitled “Fluid Channel”; a US design application entitled “Sample Plate”; and a US design application entitled “Sample Plate Carrier.”

The following examples are intended to illustrate certain embodiments of the present disclosure, but do not exemplify the full scope of the disclosure.

### EXAMPLE 1

This example illustrates various microfluidic chips and devices. The microfluidic chips can be used to culture cells in extracellular matrix (ECM) (e.g., a hydrogel) that can be used to emulate functions of specific organs (organ-on-a-chip). For instance, microfluidic chips or an array of microfluidic chips can be used in some embodiments to emulate functions of the same organ. As another example, multiple chips can be used to emulate functions of multiple organs to emulate the functions of human body more closely.

In this example, the microfluidic chips are made of thermoplastics such as polystyrene (although other materials may also be used). The example microfluidic chips may contain microstructures that facilitate 3D cell culture growth and function, which can include micrometer-sized ECM channels that contain a localized extracellular matrix (e.g., a hydrogel). The hydrogel offers cells a cellular microenvironment that closely mimics their physiological conditions that allow cell-to-ECM and/or cell-to-cell interactions through the ECM. In some cases, the cells can be a well-characterized cell line, primary cells from a donor, patient or animal source, induced pluripotent stem cells (iPSC), or the like. As another example, the cells can be cell co-cultures that include cells stabilized by fibroblasts, stromal, and/or immunological cells. Other cells can also be used.

Adjacent to and interfaced with the ECM channels are one or more media channels which can be used to allow perfusion of media into the ECM channels (e.g., containing a hydrogel or other ECM), for instance, one or more types of cell media to sustain cell culture. In some cases, the perfusion may be continuous. The perfusion of media may be used to supply oxygen and/or nutrients to the cells, and/or provide relevant mechanical cues (e.g., shear stress) to the cells in the ECM. In some cases, the media may be used to remove waste from the cells.

One non-limiting example of the microfluidic design is shown in FIG. 2. The microfluidic chips in this example contain an ECM inlet and outlet to allow the flow of ECM to be added (e.g., a hydrogel), and one or more media inlets and outlets to allow perfusion of one or more types of media. The microfluidic chips may also contain, in some cases, an optical window to allow monitoring of the cells, e.g., by high content imaging. FIG. 2 shows an example of one design of the microfluidic chips showing four common interconnect regions. Each one in this figure has one ECM channel and one medium perfusion channel. In this figure, 1 and 2 indicate the ECM inlet and outlet, respectively, 3 and 4 indicate the media inlet and outlet, respectively, and 5 indicates the common interconnect region.

Cells can be seeded, e.g., into the hydrogel and/or into a media channel to adhere to the hydrogel. The cells may include, for example, a single cell type or multiple cell types, e.g., from a co-culture.

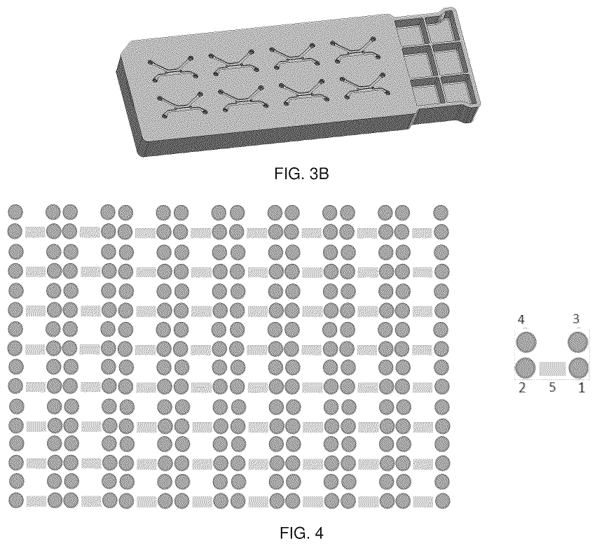

In some cases, the microfluidic devices may take the physical form of a microscope slide or a multiwell plate, or other forms. In some cases, there may be an array of microfluidic channels present. See, e.g., FIGS. 3 and 4. The dimensions of the devices and spacing between the wells, in some embodiments, may match those of microscope slides or multiwell plates, etc., for instance, so the devices are compatible with standard lab equipment such as microscopes, liquid handling instruments such as automatic pipets, automatic liquid handling stations, etc., e.g., without the need for modification. In some embodiments, the bottom of the devices may be made of optically transparent thin polystyrene films that makes them easy to image.

FIG. 3A shows an example design of a microfluidic chip design in the form of a slide. Ten microfluidic chips can be arranged on the slide. The width and the length of the slide can match those of a standard microscope slide. Another version is shown in FIG. 3B with eight such “chips” present within the slide.

FIG. 4 shows that the spacing between sample inlets and outlets can be designed to match those found on a standard 384 microwell plate. The distances between the sample inlets or outlets in this example, e.g. 1 and 3, 2 and 4 can be designed to match the spacing between wells in a standard 384 microwell plate. Similarly, the distances between the sample inlet and outlet wells, e.g. 1 and 2, 3 and 4 of each chip can be designed to match those between three adjacent wells of a standard 384 microwell plate, and the distances between wells of adjacent chips, e.g. 2 of first column and 1 of the second column, or 3 of first row and 1 of the second row, can be designed to match those between adjacent wells of a standard 384 microwell plate.

In some embodiments, the perfusion of a medium through the media channel can be uni- or bi-directional, and can be intermittent or continuous. This may allow prefusion of the medium through the hydrogel to be precise or controlled. For example, in certain cases, e.g., when evaluating drug candidates, controlled delivery can be achieved through controlled media flow. In some embodiments, information about the cells can be determined by collecting media from the outlet, and analyzing it, e.g., to assess cell function. In addition, in some embodiments, information about the cells can be obtained by imaging the cells, e.g., information regarding changes in cell morphology, cell distribution, cell size, etc. Other techniques for obtaining cells are also possible.

Polystyrene (PS) is a hydrophobic thermoplastic, and PS is sometimes not compatible with either ECM or growth media. Thus, in this example, TC-treated polystyrene is demonstrated to make the microfluidic chips. Other thermoplastics, such as polymethylmethacrylate (PMAA) or polycarbonate (PC), can also be used.

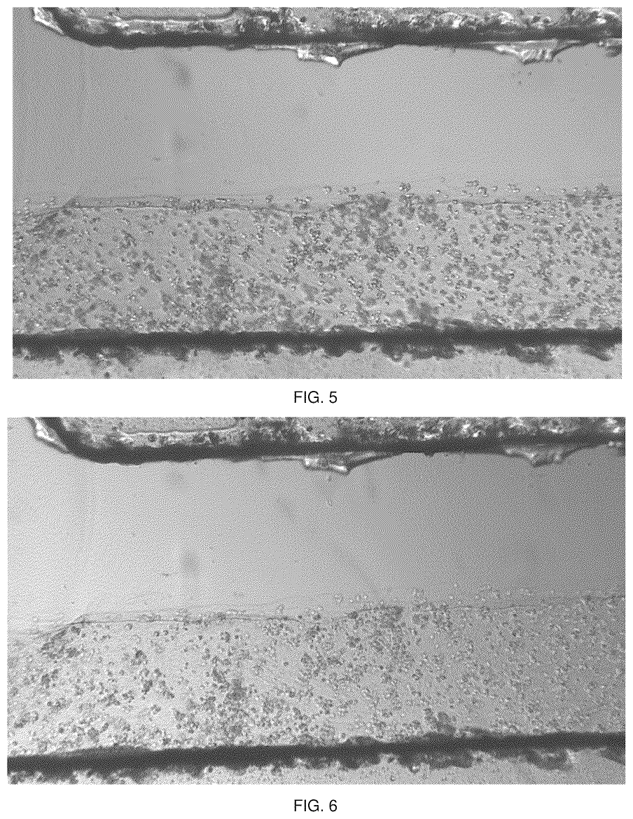

To allow the ECM to flow into the ECM channel and stay localized in the channel, additional surface treatments may be applied. Examples include oxygen or corona plasma, coating with hydrophilic materials, or the like. In some embodiments, when at least one of the surfaces that make up the ECM channels is coated with a hydrophilic polymer such as PEG or PVP, fluids containing ECM materials, such as collagen, can readily flow through the ECM channel and stay localized within the channel, even within the region where the ECM gel intersects the medium flow (see FIG. 5). Other coating materials can be used in other cases.

After gelation of the precursor, the ECM gel may be stable after multiple days of continuous perfusion of medium (FIG. 6). In one example, cells seeded in the ECM gel may be stable from continuous perfusion of medium at a flow rate of ˜100 microliters/hr for 120 hours (5 days). In addition to PVP and PEG, it was found that treating any of the surfaces of the ECM channel, e.g., by using a hydrophilic coating material that can reduce the surface tension between the hydrogel and the channel, may be conducive to localization of hydrogel in the ECM channel.

FIG. 5 shows an image of the gel/medium cross section taken after gelation of the ECM with embedded HepG2 cells. The top portion of the image is the media channel, while the bottom portion shows the ECM with embedded cells. In this figure, a clear barrierless separation between the hydrogel and the media is evident. FIG. 6 shows an image of the gel/medium cross section of the same microfluidic chip after perfusion for 24 hours at a flow rate of 100 microliters/hr. In this figure, a clear barrierless separation between hydrogel and the media is still evident.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a substrate defining a first microfluidic channel having a first inlet and a first outlet, and a second microfluidic channel having a second inlet and a second outlet, the first microfluidic channel and the second microfluidic channel positioned parallel within a common interconnect region positioned between their respective inlets and outlets;
a hydrogel filling the first microfluidic channel but not the second microfluidic channel; and
a polymer positioned between a wall of the first microfluidic channel and the hydrogel.

2. (canceled)

3. The article of claim 1, wherein the polymer comprises poly(ethylene glycol).

4. (canceled)

5. The article of any one of claim 1, wherein the polymer is hydrophilic.

6. (canceled)

7. The article of claim 1, wherein the hydrogel partially fills the common interconnect region.

8. The article of claim 1, wherein the common interconnect region has a length defined where the first microfluidic channel and the second microfluidic channel are positioned parallel of at least 2 mm.

9. The article of claim 1, wherein the first microfluidic channel and the second microfluidic channel are in fluidic contact within the common interconnect region.

10. (canceled)

11. The article of claim 1, wherein the common interconnect region is substantially straight.

12. The article of claim 1, wherein the first microfluidic and second microfluidic channel define a channel axis through the common interconnect region.

13. (canceled)

15. The article of claim 12, wherein at least 20% of any cross-section of the common interconnect region relative to the channel axis is not filled with the hydrogel.

16-18. (canceled)

19. The article of claim 1, wherein at least a portion of the first microfluidic channel outside of the common interconnect region and a portion of the second microfluidic channel outside of the common interconnect region are not parallel.

20-25. (canceled)

26. The article of claim 1, wherein at least a portion of the hydrogel in the first microfluidic channel is exposed to the second microfluidic channel.

27. (canceled)

28. The article of claim 26, wherein the portion of the hydrogel exposed to the second microfluidic channel does not contain an interface material separating the hydrogel from the second microfluidic channel.

29. (canceled)

30. The article of claim 1, wherein the hydrogel comprises collagen.

31. The article of claim 1, wherein the hydrogel comprises Matrigel®.

32-38. (canceled)

39. The article of claim 1, wherein the substrate defines a plurality of repeat units, wherein at least some of the repeat units are defined by the first microfluidic channel, the second microfluidic channel, and the common interconnect region.

40. (canceled)

41. The article of claim 39, wherein the substrate comprises at least 4 repeat units.

42. (canceled)

43. The article of claim 39, wherein the substrate has dimensions of (75 mm+/−2 mm)×(25 mm+/−2 mm).

44. The article of claim 39, wherein the substrate has dimensions of (128 mm+/−5 mm)×(85 mm+/−5 mm).

45-49. (canceled)

50. An article, comprising:
a substrate defining a first microfluidic channel having an inlet and an outlet, and a second microfluidic channel having an inlet and an outlet, the first microfluidic channel and the second microfluidic channel positioned parallel within a common interconnect region positioned between their respective inlets and outlets;
a scaffold medium filling the first microfluidic channel but not the second microfluidic channel; and
a polymer positioned between the scaffold medium and the first microfluidic channel.

51-52. (canceled)

53. A method, comprising:
imaging living cells in contact with a hydrogel contained within a microfluidic channel defined by a substrate, wherein the hydrogel partially fills the microfluidic channel and is positioned relative to the microfluidic channel by a polymer; and
flowing cell media through the microfluidic channel containing the hydrogel.

54-63. (canceled)
